(19) United States
(12) Patent Application Publication
(10) Pub. No.: US 2024/0161274 A1
(43) Pub. Date: May 16, 2024

(54) System and Method for Exposure Control and Imaging Technique Optimization Employing a Preshot X-Ray Image

(71) Applicant: GE Precision Healthcare LLC, Wauwatosa, WI (US)

(72) Inventors: Ping Xue, Pewaukee, WI (US); German Guillermo Vera Gonzalez, Menomonee Falls, WI (US); Gireesha Chinthamani Rao, Pewaukee, WI (US); Hongxu Yang, Utrecht (NL); Najib Akram, Waukesha, WI (US); Zhaohua Guo, Beijing (CN)

(21) Appl. No.: 17/985,650

(22) Filed: Nov. 11, 2022

(51) Int. Cl. G06T 7/00; G06V 10/24; G06V 10/25

(52) U.S. Cl. CPC G06T 7/0012; G06V 10/24; G06V 10/25; G06T 2207/10116; G06T 2207/10144; G06V 2201/07

(57) ABSTRACT

According to one aspect of an exemplary embodiment of the disclosure, a system and method for determining the location of one or more regions of interest (ROIs) within one or more preshot images taken of an anatomy includes the steps of providing an imaging system having a radiation source, a detector, and a camera aligned with the detector. A control processing unit is operably connected to the radiation source, the detector and the camera to generate preshot images and a camera image(s) of the subject. The camera image and preshot images are employed to determine the location of one or more regions of interest (ROIs) within the preshot images, such that exposure parameters for the operation of the radiation source to obtain one or more main shots of the subject can be adjusted corresponding to the image data for the ROI from the one or more preshot images.

### FIELD OF THE DISCLOSURE

The subject matter disclosed herein generally relates to X-ray imaging systems. More specifically, the subject matter relates to systems and methods for determining X-ray exposure parameters.

### BACKGROUND OF THE DISCLOSURE

X-ray systems, such as digital radiography (RAD) systems, mammography systems, computed tomography systems, and the like are used to generate images showing internal features of a subject. In the medical context, such systems are used for viewing internal anatomies and tissues, such as for diagnostic purposes. In modern projection X-ray systems, for example, X-rays are generated by an X-ray source and are directed towards a patient or other subject. The X-rays transfer through the subject, and are absorbed or attenuated by internal features. The resulting X-rays impact a digital detector where image data is generated. Collecting the image data allows for reconstruction of a useful image. Similar techniques are used for mammography, computed tomography, fluoroscopy and tomosynthesis image generation.

It is a general goal in radiography to acquire sufficient image data for reconstruction of a useful image, while optimizing, and often minimizing the dosage of radiation to the patient. Various techniques have been developed for estimating or controlling the imaging process to obtain these goals. The exposure parameters, e.g., peak kilovoltage and milliampere second values, that define the X-ray beam generated by the X-ray source directed towards the patient are normally either determined manually by default protocol selection or further adjusted by an operator. Exposure time could also be controlled by a computing device using, for example, automatic exposure control (AEC) methods.

The technique of manual determination employs a fixed time exposure using parameters manually defined by the operator, and as such is very dependent on operator skill and on the subjective estimation of the exposure needed to realize a clinically useful image and image quality. However, manual methods often generate low quality images (e.g., images with low signal to noise ratio), precisely because the determination of the exposure parameters is subjective. Further, in addition to producing low quality images that often require a retake, the manual method can result in over-exposure of the area or region of interest (ROI) within the patient. In either situation, or when both situations occur, the result is an unnecessary increase of the radiation dose to the patient, which is highly undesirable.

In an attempt to minimize the radiation dose to a patient, with regard to the operation of ACE systems and methods, a computing device controls the exposure parameters based on information received from sensors in the form of one or more dose sensors, such as ion or ionization chambers or solid-state sensors, coupled with the X-ray detector which measure radiation exposure. Current digital radiography systems using AEC or photo-timing to control exposure (and consequently dose) to the patient rely on proper alignment of patient anatomy with respect to fixed locations on the digital RAD system, i.e., the positions on the detector where the sensors/ionization chambers are located.

Problems arise, however, in situations where it is difficult to align body parts with the fixed locations on the system, especially when these fixed locations or sensors are not properly adapted to the patient anatomies, patient sizes, and so forth. By way of example, pediatric imaging is especially challenging because it is often difficult to align smaller body parts with the ion chambers of the imaging system. Extremity imaging, both adult and pediatric, faces similar challenges. Because the exposure measurement devices, such as ion chambers, serve as integrators of received radiation, misalignment of the anatomy being imaged relative to the ionization chambers/sensors may result in under or over-estimating the radiation actually applied to the anatomy or region of interest (ROI).

Further, certain radiography and digital radiography (RAD) systems, such as mobile RAD systems, cannot employ AEC systems and methods due to the lack of ionization chambers associated with the detector in these mobile RAD systems.

In addition, on many occasions the RAD system is utilized to obtain multiple images of a patient anatomy that are subsequently stitched or pasted together, i.e., an image pasting process, in order to form a larger image for diagnostic purposes. The differences in the shape of the different portions of the anatomy to be imaged create difficulties with regard to the alignment of those different portions of the anatomy with the sensors/ionization chambers utilized with AEC systems and methods, such as described previously.

Further, due to a thickness variation across different portions of anatomy being imaged, it is not optimal to use a single fixed technique for all acquisitions due to the differences between the ROIs, i.e., the thickness of the ROIs and/or the surrounding tissues across the anatomy being imaged. To avoid dose variation to the ROIs, typically AEC is used in order to determine the exposure parameters for the ROIs. However, AEC sensors have fixed locations which are often shifted from the positions of the ROIs in the various portions of the anatomy being imaged, such as shown in FIG. 1, where the position of the anatomy 202 relative the AEC sensors/ionization chambers 204. This, in turn, causes dose variation for the ROIs in the image(s), particularly with regard to image pasting processes, and the resulting images of the ROIs can also be affected by raw radiation if one or more sensors/ionization chambers for the AEC is not covered by a portion of the anatomy.

As an alternative to either manual or AEC systems and methods for exposure optimization, the use of a preliminary low dose X-ray image, i.e., a preshot, can be utilized to determine the imaging parameters. One of advantages with preshot is the automatic identification of anatomical area or area or region of interest (ROI) in anatomy on the image from the preshot. Then, based on signal estimated from those regions of the detector in alignment with the ROI, an appropriate technique and/or imaging parameters can be set for main shot/X-ray image to achieve desired dose level and image quality for main shot image. One example of a suitable process of this type is disclosed in U.S. Pat. No. 6,795,526, entitled Automatic Exposure Control For A Digital Image Acquisition System, the entirety of which is expressly incorporated by reference herein for all purposes.

However, due to the short time between exposures in RAD systems, and in particular regard to the timing of the images obtained in the process of image pasting performed with RAD systems required to minimize movement of the patient between images, the ability to use prior art preshot techniques in image pasting processes on RAD systems is not applicable.

Therefore, with regard to each of the aforementioned shortcomings of prior art imaging systems concerning the ability of those imaging systems to detect ROIs and provide exposure parameters for effective imaging of the ROIs in image pasting processes performed on RAD systems, it is desirable to develop an improved system and method for the detecting the ROIs and providing appropriate exposure parameters with the speed necessary to accommodate an image pasting process on a RAD system.

### SUMMARY OF THE DISCLOSURE

According to one aspect of an exemplary embodiment of the disclosure, a method for determining the location of one or more regions of interest (ROIs) within one or more preshot images taken of an anatomy includes the steps of providing an imaging system having a radiation source, a detector alignable with the radiation source, the detector having a support on or against which a subject to be imaged is adapted to be positioned, a camera aligned with the detector, a control processing unit operably connected to the radiation source and detector to generate image data in an imaging procedure performed by the imaging system, and to the camera to generate camera images, the controller including a central processing unit and interconnected database for processing the image data from the detector to create preshot images, a display operably connected to the controller for presenting information to a user, and a user interface operably connected to the control processing unit to enable user input to the control processing unit, positioning the subject between the radiation source and the detector, operating the radiation source and detector to generate one or more preshot images, operating the camera to generate a camera image, determining a location of an ROI within at least one of the camera image and the one or more preshot images, and adjusting exposure parameters for the operation of the radiation source to obtain one or more main shots of the subject corresponding to the image data for the ROI from the one or more preshot images.

According to another aspect of an exemplary embodiment of the disclosure, a radiography imaging system includes a radiation source, a detector alignable with the radiation source, a camera alignable with the detector, a control processing unit operably connected to the radiation source, the detector and the camera to generate image data and camera images, the control processing unit including image processing circuitry and an interconnected database for processing the image data from the detector, a display operably connected to the controller for presenting information to a user and a user interface operably connected to the controller to enable user input to the controller, wherein the image processing circuitry is configured to determine a location of an ROI within at least one of the camera image and the one or more preshot images of a subject, and to adjust exposure parameters for the operation of the radiation source to obtain one or more main shots of the subject corresponding to the image data for the ROI from the one or more preshot images.

These and other exemplary aspects, features and advantages of the invention will be made apparent from the following detailed description taken together with the drawing figures.

### BRIEF DESCRIPTION OF THE DRAWINGS

These and other features, aspects, and advantages of the present invention will become better understood when the following detailed description is read with reference to the accompanying drawings that illustrate the best mode currently contemplated of practicing the present disclosure and in which like characters represent like parts throughout the drawings, wherein:

FIG. 1 is a schematic view of a prior art pasted image including locations of sensors/ionization chambers on an associated detector.

FIG. 2 is a diagrammatical representation of an X-ray imaging system designed to permit specification of a region of interest and translation of the region of interest into the system coordinates for X-ray exposure control according to an exemplary embodiment of the disclosure.

FIG. 3 is illustrating a method of operation of the X-ray imaging system of FIG. 2 to determine optimized X-ray imaging exposure parameters for one or more regions of interest (ROIs) in an anatomy to be imaged according to an exemplary embodiment of the disclosure.

FIG. 4 is a schematic view of a camera image obtained by the X-ray system of FIG. 2 including an indication of ROIs therein according to an exemplary embodiment of the disclosure.

FIG. 5 is a schematic view of a number of preshot images obtained by the X-ray system of FIG. 2 forming a pasted image according to an exemplary embodiment of the disclosure.

FIG. 6 is a schematic view of the preshot images and pasted image of FIG. 5 including a representation of the ROIs identified in the camera image of FIG. 4 according to an exemplary embodiment of the disclosure.

FIGS. 7A-7E are schematic views of a method of indication of ROIs in the camera image and the preshot images/pasted image according to an exemplary embodiment of the disclosure.

FIGS. 8A-8B are schematic views of a method of indication of ROIs in the camera image and the preshot images/pasted image according to another exemplary embodiment of the disclosure.

FIG. 9 is a schematic view of a method of indication of ROIs in the camera image and the preshot images/pasted image according to still another exemplary embodiment of the disclosure.

FIGS. 10A-10C are schematic views of a method of indication of ROIs in the camera image and the preshot images/pasted image according to a further exemplary embodiment of the disclosure.

FIGS. 11A-11B are schematic views of a method of indication of ROIs in the camera image bounded by an imaging boundary for the preshot images/pasted image according to a further exemplary embodiment of the disclosure.

FIGS. 12A-12B are schematic views of a method of automatic indication of ROIs in the preshot images/pasted image according to an exemplary embodiment of the disclosure.

FIG. 13 is a schematic view of an AI model trained to automatically locate ROIs within preshot images supplied to the AI model according to another exemplary embodiment of the disclosure.

FIG. 14 is a schematic view of a preshot image of an anatomy, and a corresponding thickness map and ROI map generated by the AI model of FIG. 13 according to an exemplary embodiment of the disclosure.

FIG. 15 is a schematic view of a preshot image of another anatomy, and a corresponding thickness map and ROI map generated by the AI model of FIG. 13 according to an exemplary embodiment of the disclosure.

FIG. 16 is a schematic view of a preshot image of a further anatomy, and a corresponding thickness map and ROI map generated by the AI model of FIG. 13 according to an exemplary embodiment of the disclosure.

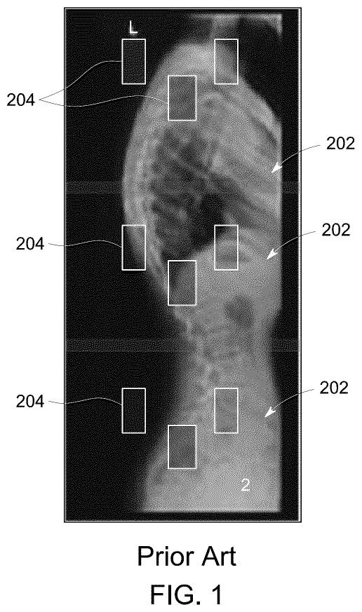

### DETAILED DESCRIPTION OF THE DRAWINGS

One or more specific embodiments will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present invention, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

As used herein, “electrically coupled”, “electrically connected”, and “electrical communication” mean that the referenced elements are directly or indirectly connected such that an electrical current may flow from one to the other. The connection may include a direct conductive connection, i.e., without an intervening capacitive, inductive or active element, an inductive connection, a capacitive connection, and/or any other suitable electrical connection. Intervening components may be present.

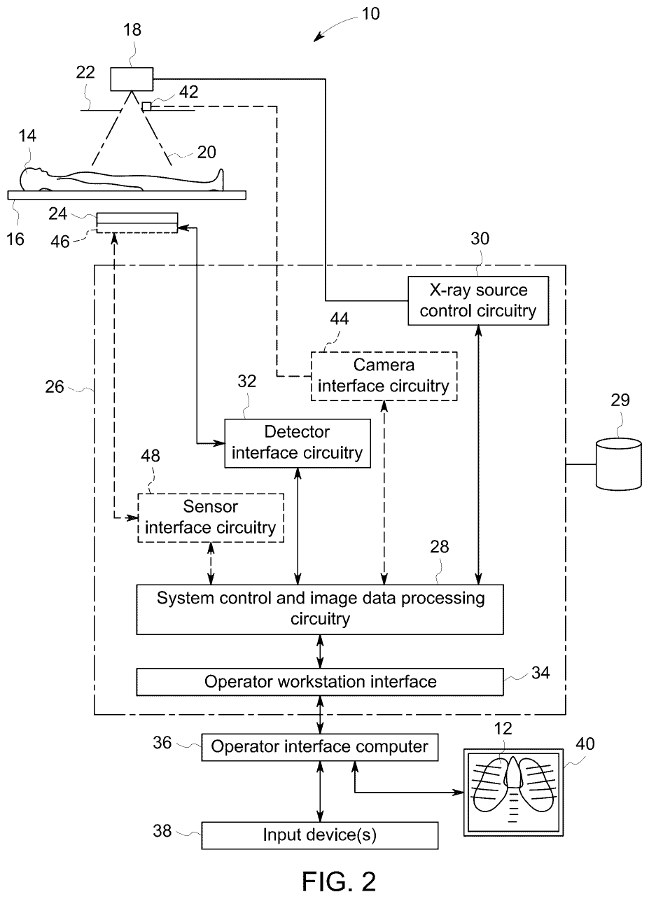

Referring to FIG. 2, an X-ray imaging system 10 is illustrated that allows for identification of a region of interest and exposure control based upon the region of interest. The X-ray imaging system 10, such as that disclosed in US Patent Application Publication No. 2012/0128125, entitled Region Of Interest Determination For X-Ray Imaging, which is expressly incorporated herein by reference for all purposes, is adapted for generating images 12 of a subject 14. In a medical diagnostic context, the subject 14 may be positioned on a support 16. An X-ray source 18 is adapted to produce a beam of radiation 20 which passes through collimator 22. The radiation traverses the subject, with some of the radiation being attenuated or absorbed, and resulting radiation impacting a detector 24. Alternatively, the system 10 can be a mobile RAD system, and/or the subject 14 can be located in a standing position in front of the detector in a digital radiography (RAD) imaging system as disclosed in US Patent Application Publication No. 2021/0183055, entitled Methods And Systems For Analyzing Diagnostic Images, which is expressly incorporated herein by reference in its entirety for all purposes.

A control processing system/processing unit/processor 26 is coupled to both the radiation source 18 and the detector 24. In general, this system 26 allows for regulation of operation of both the source 18 and the detector 24, and permits collection of information from the detector 24 for reconstruction of useful images. In the illustrated embodiment, for example, the control and processing unit/system 26 includes system control and image processing circuitry 28. Such circuitry 28 will typically include a programmed processor, supporting memory/database 29, specific applications executed by the processor during operation, which may be stored in memory/dataset 29 along with executable instructions for the operation of the control processing unit 26, and so forth. The circuitry 28 will be coupled to X-ray source control circuitry 30 that itself allows for control of operation of the X-ray source 18. The X-ray source control circuitry 30 may, for example, under the direction of the system control and image data processing circuitry 28, regulate the current and voltage applied to the X-ray source 18, alter the configuration of the collimator 20, trigger the generation of X-rays from the source 18, trigger startup and shutdown sequences of the source, and so forth.

The system control and image data processing circuitry/processing unit/processor 28 is further coupled to detector interface circuitry 32. This circuitry 32 allows for enabling the digital detector 24, and for collecting data from the digital detector 24. As will be appreciated by those skilled in the art, various designs and operations of such detectors 24 and detector interface circuitry 32 are known and are presently in use. Such designs will typically include detectors 24 having an array of discrete pixel elements defined by solid state switches and photodiodes. The impacting radiation affects the charge of the photodiodes, and the switches allow for collection of data/information regarding the impacting radiation (e.g., depletion of charge of the photodiodes). The data/information may then be processed to develop detailed images in which gray levels or other features of individual pixels in an image are indicative of the radiation impacting corresponding regions of the detector 24.

The control processing unit 26 is also illustrated as including an operator workstation interface 34. This interface allows for interaction by an operator who will typically provide inputs through an operator interface computer 36. The operator interface computer 36 and/or the system control and image data processing circuitry 28 may perform filtering functions, control functions, image reconstruction functions, and so forth. One or more input devices 38 are coupled to the operator interface computer 36, such as a keyboard, a stylus, a computer mouse, combinations thereof, among other suitable devices. The operator interface computer 36 is further coupled to a display or monitor 40 on which images may be displayed, instructions may be provided, regions of interest (ROIs) may be defined as discussed below, and so forth. In general, the operator interface computer 36 may include memory and programs sufficient for displaying the desired images, and for performing certain manipulative functions, in particular the definition of a region of interest (ROI) for image exposure control.

It should be noted that, while through the present discussion reference is made to an X-ray system 10 in the medical diagnostic context, the present invention is not so limited. For example, the invention may be used for other radiological applications, such as fluoroscopy, computed tomography, tomosynthesis and so forth. The system 10 may be used in other application contexts as well, such as part and parcel inspection, screening and so forth. Moreover, in certain contexts, and certain aspects of the detectors may be used with non-digital detectors, such as conventional film.

The system illustrated in FIG. 2 is adapted to allow for selection or definition of a region of interest (ROI) that will serve for exposure control during imaging sequences. In the particular embodiment illustrated, a camera 42 may be positioned above the patient and coupled to camera interface circuitry 44. It is contemplated that the camera 42 may be used to generate one or more images 46 of the subject 14 that can form the basis for operator definition of a region of interest (ROI) as described below. The camera 42 can have any suitable form, such as any one or more of an RGB camera, a black and white camera, a depth camera, and infrared camera, or an ultrasonic imaging camera or device. The camera interface circuitry 44 allows for triggering the camera 42 to collect a camera image data/image(s) 46 that can be processed by the camera interface circuitry 44 and forwarded to the system control and image data processing circuitry 28. The image may then be conveyed to the operator interface computer 36 and displayed on the monitor 40.

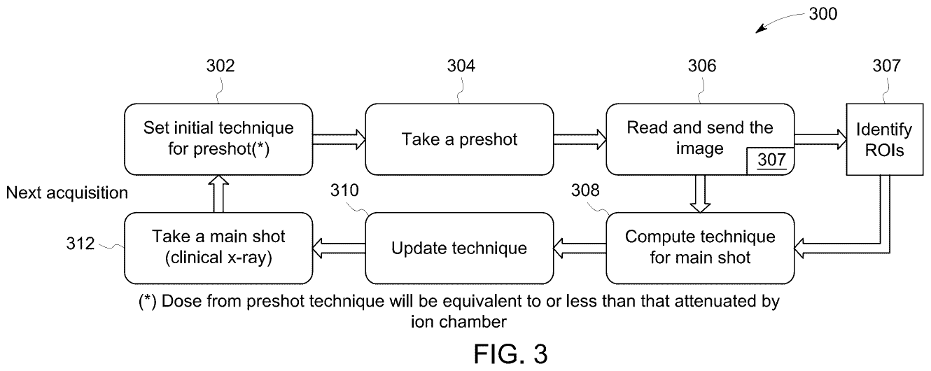

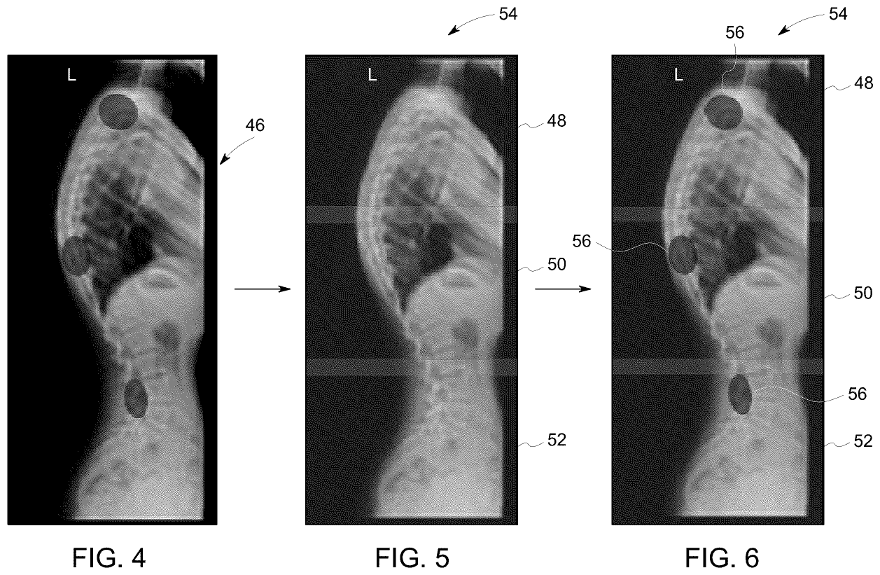

FIG. 3 schematically illustrates the method 300 employed to obtain the exposure parameters for an image pasting procedure performed using the system 10. In an initial step 302, the system 10 sets an exposure parameter or technique for obtaining one or more preshots 48,50,52 (FIG. 4) of the subject 14, with the radiation dose from the one or more preshots 48,50,52 being equivalent to or less than the dose from system employing prior art AEC systems. In step 304, the one or more preshots 48,50,52 are taken using the predetermined techniques, and in step 306 the image data from the detector 24 is transmitted to and read by the image data processing circuitry 28. In step 308, the image data processing circuitry 28 determines an updated technique/exposure parameters optimized for the ROI(s) 56 (FIG. 4) for the main shot image(s) corresponding to each of the preshots 48,50,52.

Additionally, the steps 304-308 can be performed individually for each preshot 48,50,52 to be obtained, or can be performed collectively, e.g., all three preshots 48,50,52 being taken in step 304 prior to sending the image data to the image data processing circuitry 28 in step 306.

After determination of the updated technique/exposure parameters optimized for the ROI(s) 56, the image data processing circuitry 28 outputs this updated technique to the control and processing system 26 in step 310. This updated technique/exposure parameters are then employed by the control and processing system 26 in step 312 to obtain the main shot image(s). After obtaining the main shot image(s) in step 312, the method 300 can terminate, or can reset to step 302 to prepare to obtain another set of one or more preshots 48,50,52.

With regard to the steps 308 and 310 of the method 300, after the determination of the location and form, e.g., shape and thickness, of the ROIs 56,70 in the preshot images 48,50,52, the image data processing circuitry 28 can optimize the parameters and/or technique for the main shot images to be taken corresponding to the preshot images 48,50,52 including each of the ROIs 56,70. The optimization of the parameters and technique based on the data from the preshot images 48,50,52 can include, but is not limited to kVp, mA, ms, filter, and the FOV for each corresponding main shot, with the image data processing circuitry 28 configured to automatically adjust any one or more of these parameters.

Referring now to FIG. 4, an image 46 of the subject 14 that is obtained by the camera 42 is illustrated. The image 46, which can be obtained prior to, simultaneously with or after obtaining the preshots 48,50,52, presents the exterior of the subject 14 as positioned on the support 16 adjacent the detector 24, such that the position of the subject 14 relative to the detector 24 is shown. FIG. 5 illustrates a number of individual preshot X-ray images 48,50,52 taken of the subject 14 that correspond to the camera image 46, such that the orientation of the subject 14 in the preshot images 48,50,52 relative to the detector 24 is substantially similar or identical to that in the camera image 46. Alternatively, the image data processing circuitry 28 can employ a suitable image registration process to register the camera image 46 into alignment with the preshot images 48,50,52. The preshot images 48,50,52 are illustrated in a pasted or stitched configuration in order to provide a single pasted image 54 of the entire anatomy of the subject 14 being imaged.

In order to determine the optimized exposure parameters for one or more regions of interest (ROI) 56 on the subject 14 according to step 308 in the method 300, in one exemplary embodiment, as an integral part of step 306 or as a separate step 307 (FIG. 3), the ROI(s) 56 are identified within the preshot images 48,50,52. In one exemplary embodiment of performing this identification, the camera image 46 is presented on the display 40, as shown in FIG. 4. The user can then indicate directly on the camera image 46 the location of the ROI(s) 56 on the subject 14. With this information concerning the ROI(s) 56 on the camera image 46, the alignment of the camera image 46 with the preshot images 48,50,52/pasted image 54 enables the representation of the location of the ROI(s) 56 within the preshot images 48,50,52/pasted image 54, providing the indication of the ROI(s) 56 within the preshot images 48,50,52/pasted image 54 as shown in FIG. 6. In addition, the information provided by the preshot images 48,50,52 regarding the attenuation of the X-rays emitted from the source 18 that pass through the ROI(s) 56 prior to contacting the detector 24 enables the image data processing circuitry 28 to determine the necessary and/or optimal exposure parameters for a subsequent main shot of the subject 14 to be performed by the control and processing system 26. Further, the subsequent main shot or shots taken of the ROIs 56 identified in the preshot images 48,50,52 can be obtained in the same manner or a different manner as the preshot images 48,50,52, e.g., the main shot images can be obtained as completely separate images or can be obtained and pasted together to form a pasted main shot image for diagnosis.

In an alternative embodiment, as opposed to registering the camera image 46 to the preshot images 48,50,52/pasted image 54, or in addition to the registration, the camera image 46 can be presented as an overlay directly on the preshot images 48,50,52/pasted image 54 for identification of the ROI(s) 56.

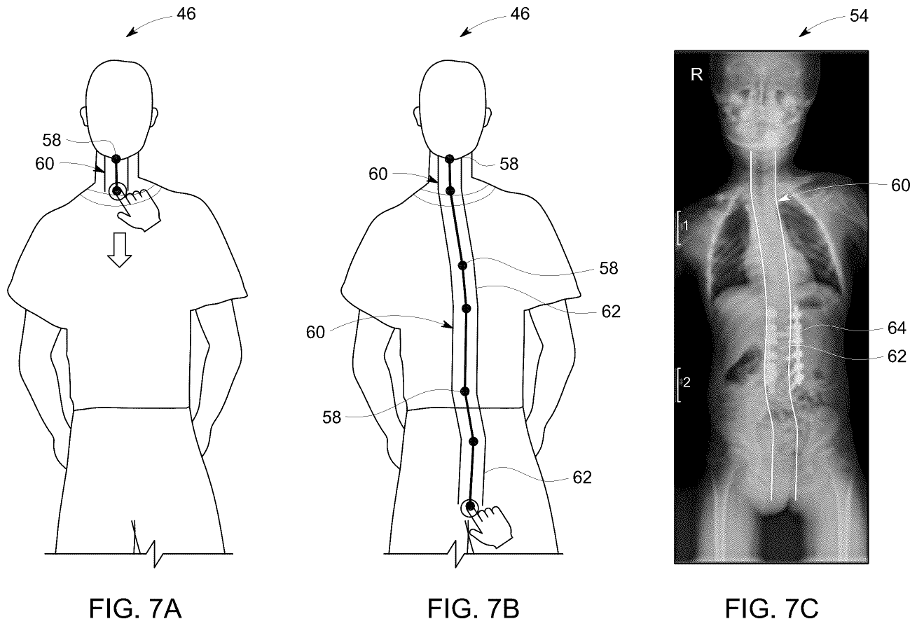

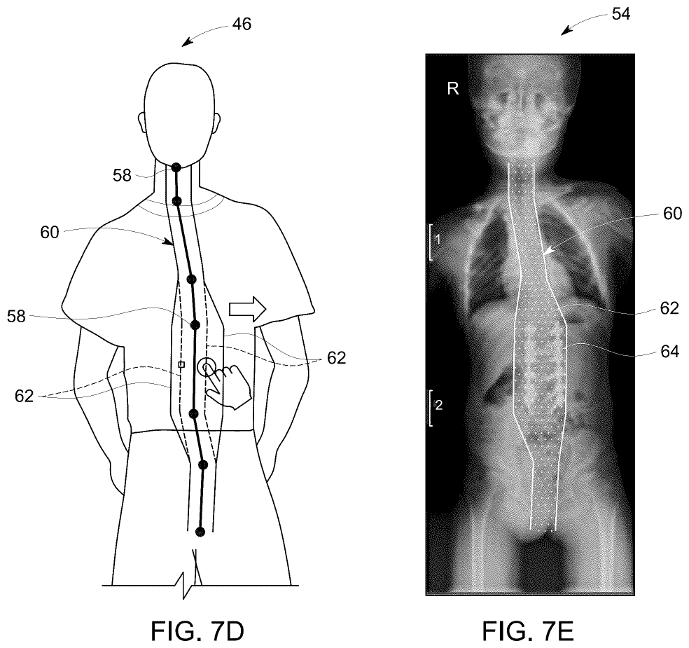

With regard to the manner in which the ROI(s) 56 are identified on the images 46,54 in step 306/307, referring now to FIGS. 7A-7E, in FIG. 7A a camera image 46 of the subject 14 is presented on the display 40. Using the input device 38, the operator can create or draw the location of the ROI(s) 56 directly on the camera image 46. In the illustrated exemplary embodiment of FIG. 7B, the operator can provide or select a number of points 58 on the camera image 46 representative of the ROI(s) 56. The one or more points 58 can each be designated as individual and separate ROI(s) 56 within the camera image 46, or as shown in FIG. 7B, can be designated as points 58 located along on or more continuous area(s) 60 to be selected as an ROI(s) 56. The operator can select successive points 58 within the camera image 46, where the image data processing circuitry 28 designates a line between each pair of adjacent points 58 to define the area 60 within the camera image 46. The image data processing circuitry 28 can also include a predetermined and/or manually selectable space 62 around each selected point 58 to define the area 60. This space 62 can be extended between adjacent points 58 to define the area 60.

After or simultaneously with the designation of the point(s) 58 and/or area(s) 60 in the camera image 46 by the operator, the point(s) 58 and/or area(s) 60 can be applied to the preshot images 48,50,52/pasted image 54 as shown in FIG. 7C due to the prior alignment and/or registration of the camera image 46 with the preshot images 48,50,52/pasted image 54. The representation of the point(s) 58 and/or area(s) 60 in the camera image 46 are presented within the preshot images 48,50,52/pasted image 54 on the display 40 adjacent the camera image 46 to provide a direct visualization to the operator of the locations of the point(s) 58 and/or area(s) 60 within the preshot images 48,50,52/pasted image 54

In addition, as best shown in FIG. 7D, if the space 62 defining the ROI(s) 56 for a point 58 or area 60 does not extend completely over a feature 64 represented in the preshot images 48,50,52/pasted image 54 that is desired to be included within the ROI(s) 56, the operator can adjust the space 62 to include the feature 64 within the space 62. The adjustment of the space 62 can be performed on either the camera image 46 or the preshot images 48,50,52/pasted image 54, with a corresponding adjustment made within the other image due to the registration of the camera image 46 with the preshot images 48,50,52/pasted image 54, as shown in FIG. 7E.

After the designation of the point(s) 58 and/or area(s) 60 in the above manner constituting an exemplary embodiment of step 306/307, the image data processing circuitry 28 can then determine in step 308 the optimal exposure parameter/imaging technique for the selected the point(s) 58 and/or area(s) 60 for use in updating the technique in step 310 and obtaining the main shot of the point(s) 58 and/or area(s) 60 in step 312.

Further, while the altered or updated preshot images 48,50,52/pasted image 54 including the designated point(s) 58 and/or area(s) 60 FIG. 7C is illustrated as being presented on the display 40, in alternative embodiments the updated preshot images 48,50,52/pasted image 54 can also be utilized completely internally by the image data processing circuitry 28 and the system 10 without being presented on the display 40.

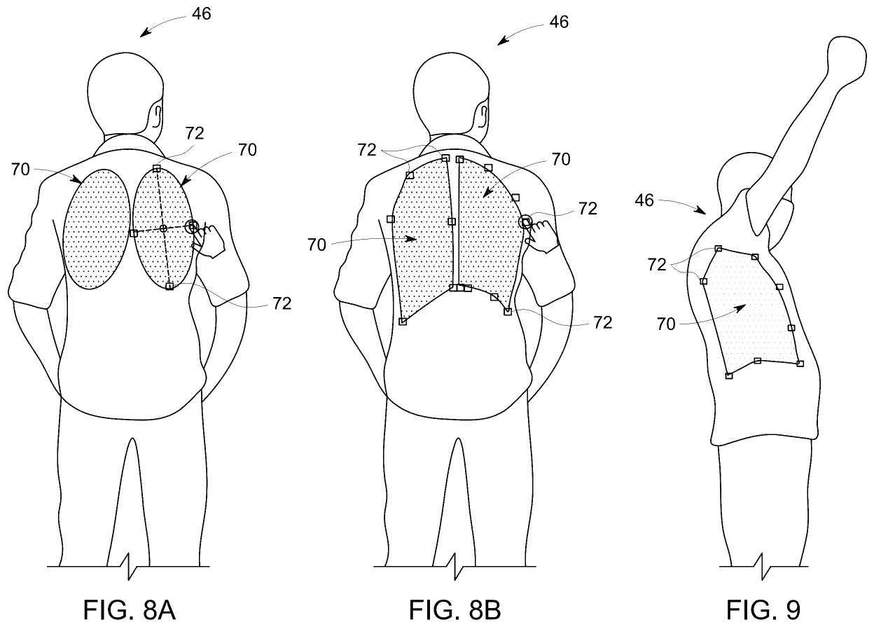

Referring now to FIGS. 8A-8B, in an alternative method for the identification of the ROI(s) 56 in steps 306/307, the designation of the specific anatomy/type of image procedure to be performed by the system 10 can provide an indication of the types of ROI(s) 56 that are normally identified within the images to be obtained. In particular, a specific anatomy/view for x-ray imaging procedure by the system 10 can have a different number of known key areas (or diagnosis areas), of different shapes and sizes that are normally employed for a determination of dose control for the anatomy/view for x-ray imaging procedure. As such, as shown in FIG. 8A, the image data processing circuitry 28 can automatically select or designate sample or system-generated ROI(s) 70 for the specific anatomy/view for x-ray imaging procedure and display the sample ROI(s) 70 on the camera image 46. The shape, size and number of sample ROI(s) 70 presented for the particular anatomy/view can be variable. For example, the shape of each ROI(s) 70 could be a circle or a square with center point, any rectangular, or regular shape with center line indicated, or even a drawn region. Further, the number of sample ROI(s) 70 presented can be determined per imaging process, such as two sample ROIs 70 in lung areas for a typical chest AP or PA imaging procedure, or one sample ROI 70 for each of a skull imaging procedure or a chest LAT imaging procedure, as shown in FIG. 9. For each sample ROI 70, the representation on the camera image 46 and/or the preshot images 48,50,52/pasted image 54 with include a number of adjustment nodes 72 spaced about the sample ROI 70, such as around the perimeter and/or along the length of the sample ROI 70. By selecting and moving an adjustment node 72, the operator can adjust the shape of the ROI 70 in order to cover the entire area desired to be encompassed within the sample ROI 70, as shown by the adjustment of the shape of the sample ROIs 70 between FIGS. 8A and 8B.

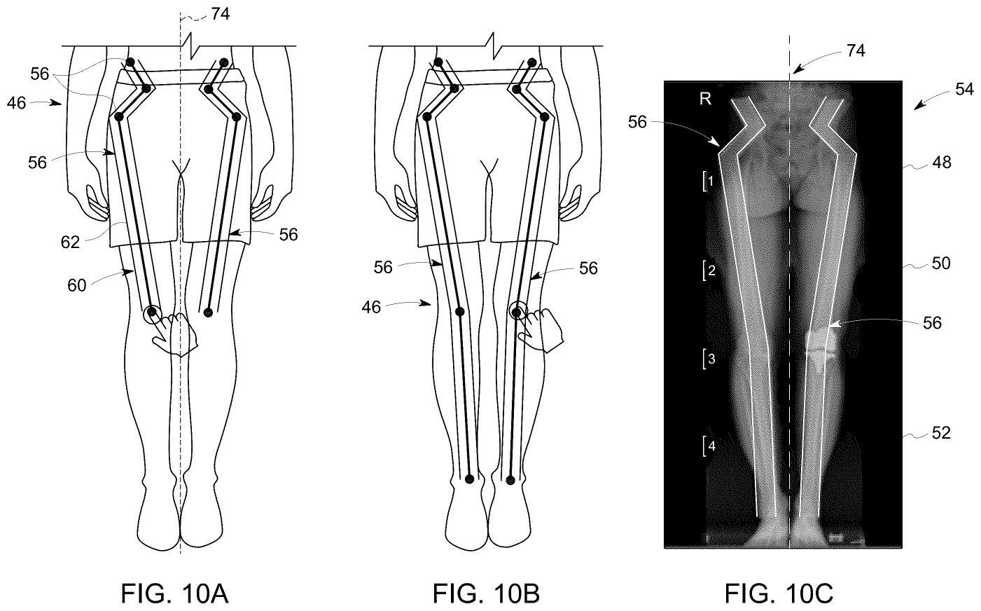

In addition, in another exemplary embodiment, for an anatomy or imaging procedure that includes a symmetrical anatomy, such as shown in FIGS. 10A-10C, when an operator defines an ROI 56 or is modifying a sample or system-generated ROI 70 provided by the image data processing circuitry 28 in step 306/307 along one side of the anatomy/subject 14 in the camera image 46, the image data processing circuitry 28 creates a mirror image ROI 56,70 on the other side of the anatomy/subject 14 using the anatomy center or the detector center as the axis of symmetry 74. For example, when creating an ROI 56 along one leg 76 of the subject 14, the image data processing circuitry 28 creates a mirror image ROI 56 along the other leg 76, which are each represented in the preshot images 48,50,52/pasted image 54 as shown in FIG. 10C. However, while the ROIs 56,70 may be initially created as mirror images of one another, the individual ROIs 56,70 can be modified together (such as to extend the length of the ROI 56,70 along the entire leg 76, as in FIGS. 10A-10B) or separately modified in order to accommodate variations in each half of the anatomy/subject 14, and/or to encompass different features present in individual halves of the anatomy/subject 14.

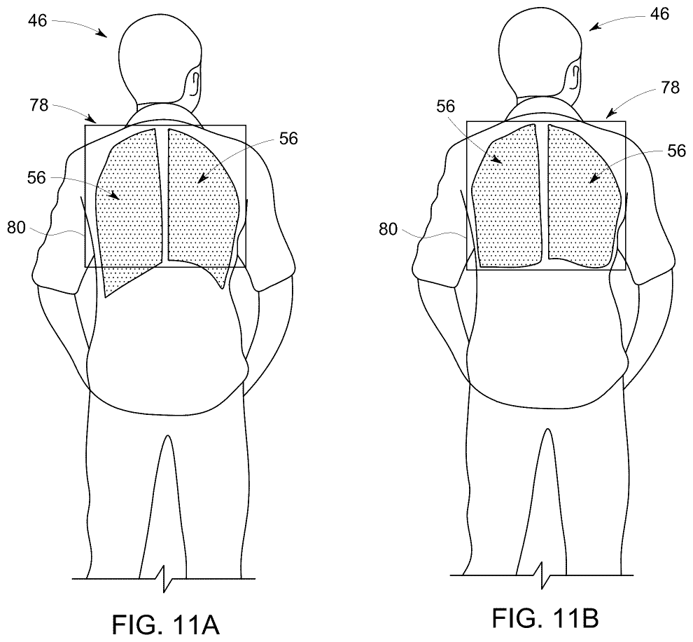

Looking now at FIGS. 11A-11B, in another exemplary embodiment for the identification of the ROIs 56,70 in step 306/307, in certain situations, the ROIs 56 selected by the operator and/or the sample ROIs 70 provided by the image data processing circuitry 28 for a particular anatomy or image view may be outside of the imaging area of the detector 24. This can occur due to the setting or position of the ROIs 56,70 being based on the camera image 46, which exceeds the imaging area of the detector 24 for each preshot image 48,50,52. In particular, after selection of the ROI 56,70 as desired, the ROI(s) 56,70 will be aligned by the image data processing circuitry 28 to the imaging area of the detector 24, based on known parameters, such as, but not limited to, collimator light, field-of-view (FOV) of the source 18 and/or camera 42, and system positioning feedback, if available. As shown in FIG. 11A, the camera image 46, whether presented independently or overlaid onto the preshot images 48,50,52/pasted image 54, will show the border 80 of the detector imaging area 78 to enable the operator to view whether any portion of the ROIs 56,70 are disposed outside the detector imaging area 78. If so, as shown in FIG. 11B, the operator can adjust the ROIs 56,70 to avoid any portion of the ROIs 56,70 being disposed outside of the imaging area 78. Further, in situations where the detector 24 is completely covered by patient/subject 14 with no positioning feedback, the selected ROI(s) 56,70 based on FOV light will be automatically adjusted by the preshot images 48,50,52 to make sure that ROIs 56,70 employed for dose computation for the main shot image are located within the imaging area 78.

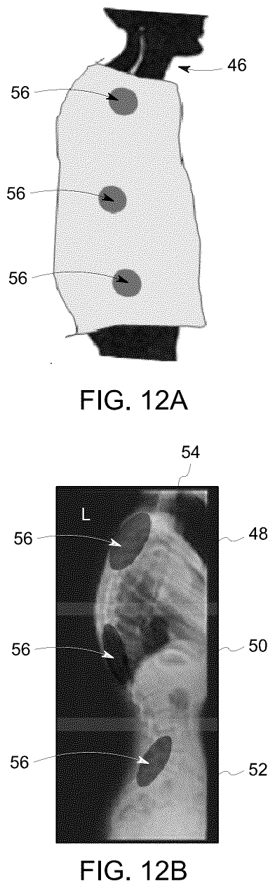

With reference now to FIGS. 12A-12B, in another exemplary embodiment of the disclosure, the selection and/or identification of the ROIs 56,70 in step(s) 306/307 can be performed automatically by the system 10, such as by the image data processing circuitry 28. In this embodiment, the image data processing circuitry 28 is provided with the information regarding the anatomy/subject 14 to be imaged and the views to be obtained in the imaging procedure, e.g., the protocol for the imaging procedure. After the system 10 has obtained the preshot images 48,50,52, the image data processing circuitry 28 can analyze the preshot images 48,50,52 in order to detect the ROIs 56,70 within the preshot images 48,50,52 using the information concerning the anatomy/subject 14 and the protocol for the imaging procedure to be employed by the system 10.

With the camera image 46 as shown schematically in FIG. 12A, in various situations the external view of the subject 14 provided by the camera image 46 may not accurately reflect the actual location of the ROIs 56,70. Thus, it can be difficult for the operator to accurately identify the ROIs 56,70 based on the camera image 46. In FIG. 12B, the preshot images 48,50,52/pasted image 54 can be analyzed by the image data processing circuitry 28 to accurately determine the ROIs 56,70 in light of the information provided from the preshot images 48,50,52, as well as from the information regarding the anatomy/subject 14 being imaged and the protocol for the main shot imaging procedure. As such, the image data processing circuitry 28 can readily and precisely identify the ROIs 56,70 in the preshot images 48,50,52 and adjust the exposure parameters accordingly for proper radiation dose control in the process of obtaining the main shot images. Further, this automatic identification system and process performed by the image data processing circuitry 28 enables operator interaction and/or selection of the ROIs 56,70 to be removed, thereby streamlining the identification process and eliminating the potential for operator error in the ROI identification procedure.

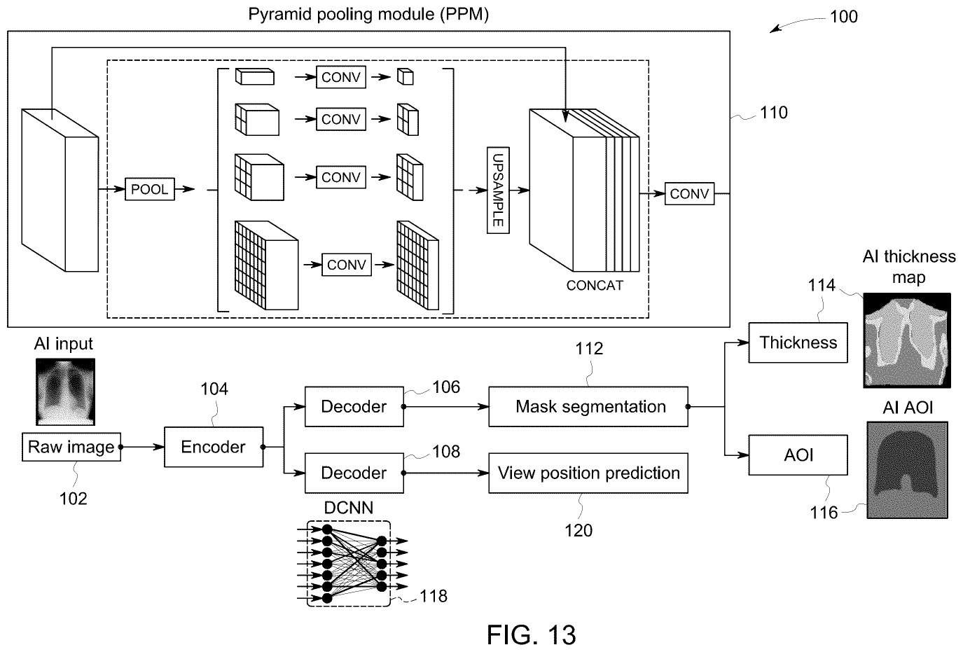

In one exemplary embodiment of the system and process or method in which the image data processing circuitry 28 can automatically identify the ROIs 56,70 in the preshot images 48,50,52/pasted image 54, referring now to FIG. 13, the image data processing circuitry 28 can include an artificial intelligence (AI) component or model 100 that operates to locate the ROIs 56,70 in the preshot images 48,50,52. The AI model 100 is trained using a dataset of preshot images having a known anatomy and a known location of the ROI 56,70 within the anatomy. In use with step 306/307 to identify the ROIs 56,70 in the preshot images 48,50,52, the trained AI model 100 can identify the ROIs for technique optimization and dose control from the preshot images 48,50,52 in conjunction with the selected protocol for the imaging procedure as well as the information provided by the camera image 46, as discussed previously. As a result, the image data processing circuitry 28/AI model 100 provides a reliable and consistent determination of the location of the ROIs 56,70 using the data form the preshot images 48,50,52 in order to determine the appropriate dose control and acquisition optimization parameters for subsequent the main shot procedure.

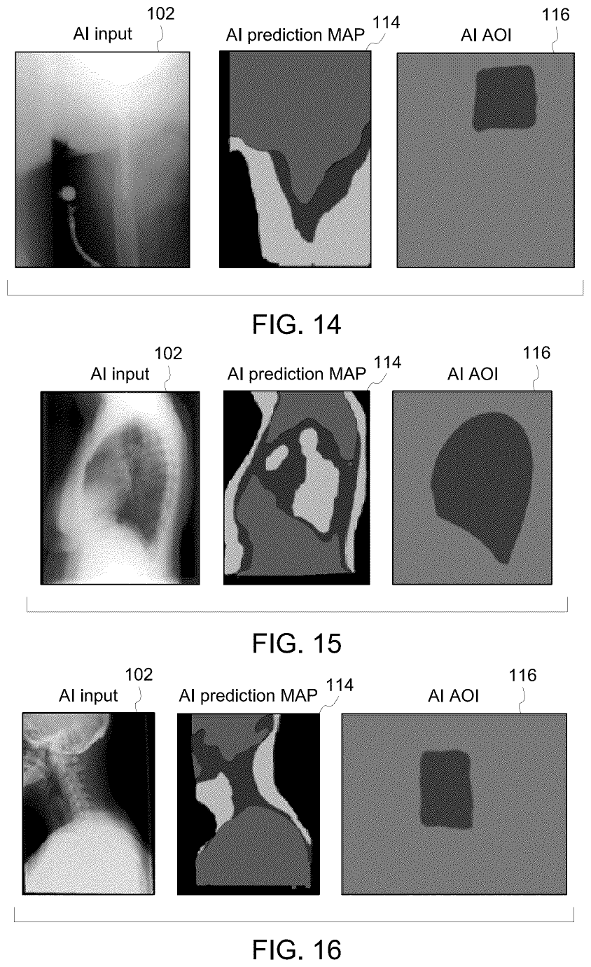

In a particular exemplary embodiment of the structure and operation of the AI model 100 in FIG. 13, after training, the AI model 100 is supplied with a preshot image/preshot image data 102 as an input. The input image/image data 102 is encoded at 104 and the encoded image/image data 104 is supplied to a first decoder 106 and a second decoder 108. The first decoder 106 can be form of a pyramid pooling module (PPM), or other decoder design, 110 and operates to provide a mask segmentation 112 of the anatomy present within the image/image data 102 that supplies a thickness map 114 and an ROI map 116 for the image/image data 102, examples of which are shown in FIGS. 14-16 for images/image data 102 of different anatomies. The second decoder 108 takes the form of a convolutional neural network (CNN) 118 that provides an ROI position prediction 120 of the ROI 56,70 within the image 102 for the particular view for the image 102. With each of the thickness map 114, ROI map 116, ROI position prediction 120 and combinations thereof, the AI model 100 can accurately locate the ROIs 56,70 within the preshot images 48,50,52 supplied to the AI model 100 in order to determine the appropriate dose control and acquisition optimization parameters for subsequent the main shot procedure.

According to another exemplary embodiment of the ROIs 56,70 can be dynamically segmented/identified per anatomy/view change during image pasting and other imaging procedures performed utilizing the imaging system 10 including the image data processing circuitry 28 and/or AI model 100. Further, the image data processing circuitry 28 and/or AI model 100 can be combined with a traditional segmentation methods to cover all applications for use of the system 10 including the image data processing circuitry 28 and/or AI model 100, including both dual energy and single energy imaging applications.

Finally, it is also to be understood that the system 10 may include the necessary electronics, software, memory, storage, databases, firmware, logic/state machines, microprocessors, communication links, displays or other visual or audio user interfaces, printing devices, and any other input/output interfaces to perform the functions described herein and/or to achieve the results described herein. For example, as previously mentioned, the system may include at least one processor and system memory/data storage structures, which may include random access memory (RAM) and read-only memory (ROM). The at least one processor of the system 10 may include one or more conventional microprocessors and one or more supplementary co-processors such as math co-processors or the like. The data storage structures discussed herein may include an appropriate combination of magnetic, optical and/or semiconductor memory, and may include, for example, RAM, ROM, flash drive, an optical disc such as a compact disc and/or a hard disk or drive.

Additionally, a software application that adapts the controller to perform the methods disclosed herein may be read into a main memory of the at least one processor from a computer-readable medium. The term “computer-readable medium”, as used herein, refers to any medium that provides or participates in providing instructions to the at least one processor of the system 10 (or any other processor of a device described herein) for execution. Such a medium may take many forms, including but not limited to, non-volatile media and volatile media. Non-volatile media include, for example, optical, magnetic, or opto-magnetic disks, such as memory. Volatile media include dynamic random access memory (DRAM), which typically constitutes the main memory. Common forms of computer-readable media include, for example, a floppy disk, a flexible disk, hard disk, magnetic tape, any other magnetic medium, a CD-ROM, DVD, any other optical medium, a RAM, a PROM, an EPROM or EEPROM (electronically erasable programmable read-only memory), a FLASH-EEPROM, any other memory chip or cartridge, or any other medium from which a computer can read.

While in embodiments, the execution of sequences of instructions in the software application causes at least one processor to perform the methods/processes described herein, hard-wired circuitry may be used in place of, or in combination with, software instructions for implementation of the methods/processes of the present invention. Therefore, embodiments of the present invention are not limited to any specific combination of hardware and/or software.

It is understood that the aforementioned compositions, apparatuses and methods of this disclosure are not limited to the particular embodiments and methodology, as these may vary. It is also understood that the terminology used herein is for the purpose of describing particular exemplary embodiments only, and is not intended to limit the scope of the present disclosure which will be limited only by the appended claims.

We claim:

1. A method for determining the location of one or more regions of interest (ROIs) within one or more preshot images taken of am anatomy, the method comprising the steps of:
a. providing an imaging system comprising:
i. a radiation source;
ii. a detector alignable with the radiation source, the detector having a support on or against which a subject to be imaged is adapted to be positioned;
iii. a camera aligned with the detector;
iv. a control processing unit operably connected to the radiation source and detector to generate image data in an imaging procedure performed by the imaging system, and to the camera to generate camera images, the controller including a central processing unit and interconnected database for processing the image data from the detector to create preshot images;
v. a display operably connected to the controller for presenting information to a user; and
vi. a user interface operably connected to the control processing unit to enable user input to the control processing unit;
b. positioning the subject between the radiation source and the detector;
c. operating the radiation source and detector to generate one or more preshot images;
d. operating the camera to generate a camera image;
e. determining a location of an ROI within at least one of the camera image and the one or more preshot images; and
f. adjusting exposure parameters for the operation of the radiation source to obtain one or more main shots of the subject corresponding to the image data for the ROI from the one or more preshot images.

2. The method of claim 1, further comprising the step of aligning the camera image with the one or more preshot images prior to determining the location of the ROI within at least one of the camera image and the one or more preshot images.

3. The method of claim 1, wherein the step of determining a location of the ROI in at least one of the camera image and the one or more preshot images comprises providing an indication of the location of the ROI within the camera image.

4. The method of claim 3, wherein the step of providing the indication of the location of the ROI within the camera image comprises providing the indication on the camera image with the user interface.

5. The method of claim 4, further comprising the step of presenting the camera image on the display.

6. The method of claim 5, wherein the step of presenting the camera image on the display comprises presenting the camera image as an overlay on the one or more preshot images.

7. The method of claim 4, wherein the step of providing the indication on the camera image comprises drawing the indication on the camera image.

8. The method of claim 7, further comprising the step of providing a corresponding indication on the one or more preshot images after drawing the indication on the camera image.

9. The method of claim 3, wherein the step of providing the indication of the location of the ROI within the camera image comprises providing a system-generated indication on the camera image.

10. The method of claim 9, further comprising the step of modifying the system-generated indication on the camera image.

11. The method of claim 10, wherein the step of modifying the system-generated indication comprises modifying the system-generated indication with the user interface.

12. The method of claim 3, wherein the step of providing an indication of the location of the ROI within the camera image further comprises providing an indication of an imaging area boundary within the camera image in conjunction with the indication of the ROI.

13. The method of claim 1, wherein the step of determining a location of the ROI in the camera image and the one or more preshot images comprises:
a. inputting the one or more preshot images to an image processing AI model; and
b. providing the indication of the location of the ROI as an output from the AI model.

14. The method of claim 12, wherein the step of providing the indication of the location of the ROI as an output from the AI model comprises providing at least one of a thickness map and an ROI map as an output from the AI model.

15. The method of claim 12, wherein the step of providing the indication of the location of the ROI as an output from the AI model comprises providing an ROI view position prediction as an output from the AI model.

16. The method of claim 1, wherein the step of adjusting exposure parameters for the operation of the radiation source comprises adjusting one or more of kVp, mA, ms, filter, positioning, and field of view for each corresponding main shot.

17. The method of claim 1, further comprising the step of obtaining one or more main shot images of the ROIs determined within the one or more preshot images after adjusting the exposure parameters.

18. The method of claim 17, wherein the step of obtaining the one or more main shot images further comprises pasting together the one or more main shot images.

19. A radiography imaging system comprising:
a. a radiation source;
b. a detector alignable with the radiation source;
c. a camera alignable with the detector;
d. a control processing unit operably connected to the radiation source, the detector and the camera to generate image data and camera images, the control processing unit including image processing circuitry and an interconnected database for processing the image data from the detector;
e. a display operably connected to the controller for presenting information to a user; and
f. a user interface operably connected to the controller to enable user input to the controller;
wherein the image processing circuitry is configured to determine a location of an ROI within at least one of the camera image and the one or more preshot images of a subject, and to adjust exposure parameters for the operation of the radiation source to obtain one or more main shots of the subject corresponding to the image data for the ROI from the one or more preshot images.

20. The radiography imaging system of claim 19, wherein the image processing circuitry is configured to receive user input to determine the location of the ROI within at least one of the camera image and the one or more preshot images.

21. The radiography imaging system of claim 19, wherein the image processing circuitry includes an AI model trained to automatically determine the location of the ROI within the one or more preshot images.
